(19) United States
(12) Patent Application Publication
(10) Pub. No.: US 2024/0042064 A1
(43) Pub. Date: Feb. 8, 2024

(54) NANO- OR MICROPARTICLE COMPRISING A POLYVINYL ALCOHOL MATRIX AND DISPERSED THEREIN, FERRITE, METHOD FOR PRODUCING THE SAME AND USES THEREOF

(71) Applicant: COMMISSARIAT A L'ENERGIE ATOMIQUE ET AUX ENERGIES ALTERNATIVES, Paris (FR)

(72) Inventors: Olivier PONCELET, GRENOBLE (FR); Antonin DURET, GIF SUR YVETTE (FR); Guénaëlle JASMIN-LEBRAS, GIF SUR YVETTE (FR); Maïkane DEROO, GIF SUR YVETTE (FR); Cécile FERAUDET-TARISSE, GIF SUR YVETTE (FR)

(21) Appl. No.: 18/361,064

(22) Filed: Jul. 28, 2023

(30) Foreign Application Priority Data
Jul. 28, 2022 (FR) 2207815

(51) Int. Cl. A61K 49/18

(52) U.S. Cl. CPC A61K 49/1854; B82Y 5/00

(57) ABSTRACT

A nano- or microparticle comprising a matrix consisting of or comprising at least one polyvinyl alcohol (PVA) and dispersed therein, ferrite, and a method for producing the same. Further, the use of these nano- or micro-particles for the preparation and the implementation of devices that can be detected by giant magnetoresistance sensors (GMR sensors) as biological diagnostic tools.

### CROSS-REFERENCE TO RELATED APPLICATION

This application claims priority to FR 2207815, filed Jul. 28, 2022, the disclosure of which is hereby expressly incorporated by reference herein in its entirety.

### TECHNICAL FIELD

This invention relates to a nano- or microparticle comprising a matrix consisting of or comprising at least one polyvinyl alcohol (PVA) and dispersed therein, ferrite, and to a method for producing the same. The present invention is also aimed at the use of these nano- or micro-particles for the preparation and the implementation of devices that can be detected by giant magnetoresistance sensors (GMR sensors) as biological diagnostic tools.

### DESCRIPTION

The clinical diagnostics is a field in which new, faster, more direct, more precise and more selective, as well as being highly efficient and inexpensive methods of analysis are in great demand. Because of their small size, their ultra-sensitive transduction and their potential integration into “lab-on-a-chip” systems, the nanotechnology-based biosensing devices are a potential candidate for meeting all the above requirements. Over the last decade, a great deal of work has been carried out on the biomagnetism and the magnetic biosensors, based on molecular processes, in particular on the application of the magnetic nanoparticles in biomedicine, on their synthesis, their functionalisation and their detection by magnetic sensors.

The magneto resistive sensors appear to be among the best candidates for meeting these criteria. Since the late 1990s, the ultra-sensitive giant magnetoresistance (GMR sensors) sensors have been excellent candidates for the magnetic detection in a wide variety of fields, including healthcare. These are highly sensitive sensors based on spin electronics. They have the advantage of having a very high detectivity (the detectivity corresponds to the magnetic field for which the signal-to-noise ratio is 1; it is expressed in T/√Hz, and allows an easy comparison of the performance of the different magnetic sensors), of the order of 50 to 200 pT/√Hz, of being inexpensive and of being easy to integrate into labs-on-a-chip.

In this field, the biological targets are magnetically labelled by magnetic nano- or micro-particles functionalised with specific antibodies directed against the biological target of interest. These magnetically marked objects are then detected dynamically (or statically) by the GMR sensors. This technology is based on the detection of magnetic markers.

In recent years, the GMR sensors have shown a great potential as sensors for detecting biological targets (cells or bacteria). The resistance of a GMR sensor varies according to the magnetic field applied to the sensor, so a magnetically marked biological object can induce a signal. Compared with the traditional optical detection, which is widely used in biomedicine, the GMR sensors allow to work with complex matrices, even opaque ones, and allow the dynamic detection of the magnetic objects one by one. On the other hand, a new biochip, based on GMR sensors arranged on either side of the microfluidic channel, allows to determine the magnetic moment of the objects detected and thus identify a large number of false positives resulting from the presence of aggregates of magnetic nanoparticles (patent FR3082620). However, the key to further increasing the sensitivity of such a detection technique is to reduce the number of magnetic nanoparticle aggregates, which still merge with the marked biological targets of interest.

Due to the advantages of the GMR materials for the magnetic field measurements, such as the high sensitivity and the fast response under a low magnetic field conditions, an increased attention has been given to the development of these GMR materials for the biosensors.

The use of magnetic particles for applications in biotechnology, pharmaceutical industry and medicine is becoming increasingly common. The magnetic particles were developed some twenty years ago, and are now enjoying a boom, closely linked to the objectives of miniaturising the analyses. Their size, ranging from nanometric to micrometric, and the fact that they can be manipulated using a simple magnetic field, make them well suited to the manipulation and to the identification of biological molecules. Many companies supply a wide variety of microbeads on whose surface it is possible to specifically graft all kinds of molecules of interest.

In the current applications, the magnetic particles used are mainly superparamagnetic, i.e. they have no magnetic remanence and the internal field is zero in the absence of an external magnetic field, which theoretically limits the aggregation phenomena.

However, most commercial magnetic nanoparticles do not have a great colloidal stability, and form aggregates during analyses performed by them. And as mentioned above, the presence of aggregates of magnetic nanoparticles causes false positives that limit the sensitivity and the specificity of the diagnostic tests. In particular, the early diagnosis by GMR sensor is extremely sensitive, as the magnetically marked biological objects can be detected one by one, but this sensitivity, as well as the specificity of the tests, is degraded by the formation of non-specific aggregates, i.e. not specifically linked to the biological target under study but only agglutinated for physico-chemical reasons.

The aim of the present invention is to provide nano- or micro-particles which avoid the aforementioned disadvantages.

One of the aims of the invention is thus to provide magnetic nano- or micro-particles with high colloidal stability, thereby allowing to avoid the problem of aggregation, particularly during early diagnosis tests involving them.

Another aim of the invention is to provide nano- or microparticles with a magnetic moment equivalent to that of the commercial beads, while allowing to avoid the problem of aggregation encountered with these commercial beads.

Another aim of the invention is to provide monodisperse nano- or microparticles, by a simple and inexpensive method, which can be easily functionalised, in particular for the preparation and the implementation of devices capable of being detected by giant magnetoresistance sensors (GMR sensors) as biological diagnostic tools.

According to a first aspect, the invention concerns a nano- or microparticle comprising a matrix consisting of or comprising at least one polyvinyl alcohol (PVA) and dispersed therein, ferrite.

By “nanoparticle”, it is meant notably, unless otherwise stated, a particle with a size from 1 to 1000 nm, in particular from 50 to 500 nm, more particularly from 100 to 350 nm.

By “microparticle”, it is meant notably, unless otherwise stated, a particle with a size from 1 to 100 μm, in particular from 1 to 10 μm, more particularly from 1 to 5 μm.

By “ferrite” it is meant in particular a compound of the formula Fe₃O₄.

By “dispersion” it is meant in particular that the ferrite is distributed in the form of a plurality of particles in the PVA matrix.

According to a particular embodiment, the molecular weight of the polyvinyl alcohol is from 10,000 to 150,000, in particular from 50,000 to 90,000.

A PVA that is too large may increase the viscosity of the medium of formation of the particle, making it very heterogeneous. If the molecular weight of the PVA is too low, it is possible for the Fe₃O₄ seeds to coalesce, resulting in a mixture of particles of varying sizes that are likely to be above one micron.

In a particular embodiment, the matrix is free of polyvinylpyrrolidone (PVP).

“PVP-free” means that the matrix does not comprise any PVP.

In a particular embodiment, the polyvinyl alcohol (PVA) is partially acetylated. In this case, it corresponds to a partially hydrolysed polyvinyl acetate (or a poly(vinyl acetate), or PVAC). More particularly, the polyvinyl alcohol (PVA) is acetylated to less than 20% (i.e. less than 20% of the —OH groups of the PVA are acetylated), for example to less than 10% or about 10%, which corresponds to a poly(vinyl acetate) hydrolysed to more than 80% (i.e. more than 80% of the —OAc groups of the PVAC are hydrolysed), for example to more than 90% or about 90%.

In another particular embodiment, the polyvinyl alcohol (PVA) is not acetylated. In this case, it corresponds to a fully hydrolysed polyvinyl acetate (or a poly(vinyl acetate), or PVAC).

According to a particular embodiment, the polyvinyl alcohol is cross-linked, for example notably on the surface of the nano- or microparticle, in particular with the aid of a crosslinking agent chosen from the following compounds: boric acid, boronic acids, boric acid esters, boronic acid esters, borax (Na₂B₄O₇·10H₂O), and aldehydes, in particular from the compounds of the following formulae: B(OR)₃, RB(OH)₂, B(OH)₃, R-CHO, in which R is chosen from aryls and heteroaryls, and the compounds comprising at least two aldehyde functions, in particular the compounds comprising two aldehyde functions, more particularly the compounds of formula H—C(═O)—(CH₂)ₙ—C(═O)—H, with n ranging from 0 to 6, for example n=0, 1, 2 or 3, the crosslinking agent being for example 3-quinoline boronic acid. When the PVA is cross-linked, it is likely to be insensitive to the presence of a poor solvent, such as ethanol, particularly with regard to its volume.

In a particular embodiment, the nano- or microparticle according to the present invention has the shape of a sphere or spheroid.

By “spheroid” it is meant in particular a particle whose shape is close to the sphere, but not perfectly spherical, and in particular that all the dimensions of the particle are between 0.9 and 1 times the largest dimension of said particle.

According to a particular embodiment, the nano- or microparticle according to the present invention has a size ranging from 50 to 1000 nm, in particular from 150 to 450 or 500 nm.

This size corresponds in particular to a nano- or microparticle in which the PVA is free of solvent, in particular water, i.e. dried overnight at 100° C.

If necessary, the size of the nano- or microparticle can be modified: it can be reduced by adding a poor solvent such as ethanol, or increased (by hydration) using a good solvent, such as water.

“Size” refers in particular to the largest dimension of the particle.

According to a particular embodiment, the at least one polyvinyl alcohol is present in the nano- or microparticle according to the present invention in an amount of 30 to 50% by mass relative to the total mass of said nano- or microparticle.

According to a particular embodiment, the nano- or microparticle according to the present invention has a density from 1.7 to 2.5 g·cm⁻³, the density being for example about 1.87 g·cm^{31 3}.

In a particular embodiment, the ferrite is in the form of crystallites.

By “crystallites” it is meant in particular domains of material with the same structure as a single crystal.

In a particular embodiment, the ferrite is in the form of particles with an average size t_{A} from 15 to 80 nm, in particular from 15 to 60 nm, more particularly from 25 to 30 nm.

By “size” it is meant in particular the diameter of the particle when it is spherical, or the largest dimension when it is not.

For example, the average size can be measured by image analysis carried out by scanning electron microscopy (SEM).

In a particular embodiment, the ferrite is in the form of particles whose size distribution has a uniformity coefficient from 0.95 to 1.

In a particular embodiment, the ferrite is in the form of particles, more than 95% of which have a size of 28 nm +/−10%.

According to a particular embodiment, the nano- or microparticle according to the present invention has a magnetic moment of between 3.10⁻¹⁵ and 5.10⁻¹⁵ Am².

According to another aspect, the present invention also relates to an assembly of nano- or micro-particles as defined above.

All the embodiments described above for nano- or micro-particles can also be applied here, alone or in combination.

According to a particular embodiment, the size distribution of the nano- or microparticle assembly of the present invention has a uniformity coefficient from 0.95 to 1.

According to a particular embodiment, the size distribution of the assembly of nano- or microparticles of the present invention is such that more than 95% of these nano- or microparticles have a size of 180 nm +/−10%.

According to another aspect, the present invention also relates to a method for preparing a nano- or microparticle as defined above, or a nano- or microparticle assembly as defined above, comprising:
(i) a step of contacting at least one polyvinyl alcohol (PVA) with a ferrite precursor composition A to obtain a composition B;
(ii) a step of heating the composition B to obtain said nano- or microparticles.

According to a particular embodiment, the composition A comprises an iron (III) salt, optionally hydrated.

According to a more particular embodiment, the composition A comprises a compound chosen from FeCl₃, Fe(NO₃)₃, Fe(ClO₄)₃, which are optionally hydrated, the compound preferably being chosen from FeCl₃, the hydrated Fe(NO₃)₃ salts and the hydrated Fe(ClO₄)₃ salts.

In particular, the composition A comprises said iron (III) salt, optionally hydrated, and is devoid of iron (II) salt, hydrated or not.

According to a particular embodiment, the composition A comprises urea or hexamethylenetetramine, in particular urea.

In a particular embodiment, the composition A comprises a water-soluble iron chelating agent.

Said water-soluble iron chelating agent is notably chosen from citric acid, citrates, lactic acid, lactates, tartaric acid and tartrates.

According to a particular embodiment, the composition A comprises urea or hexamethylenetetramine, in particular urea, and a water-soluble iron chelating agent, in particular chosen from citric acid, citrates, lactic acid, lactates, tartaric acid and tartrates.

According to a particular embodiment, the contacting step (i) is carried out in the presence of water, the composition B obtained at the end of said step (i) being in particular an aqueous solution.

In a particular embodiment, the heating step (ii) is carried out in an autoclave.

In a particular embodiment, the heating step (ii) is carried out at a temperature from 180 to 230° C., and/or for a period from 6 to 24 hours.

According to a particular embodiment, subsequent to step (ii), and if appropriate prior to step (iii) as described below, the at least one polyvinyl alcohol (PVA) is cross-linked, in particular with the aid of a crosslinking agent chosen from the following compounds: boric acid, boronic acids, boric acid esters, boronic acid esters, borax (Na₂B₄O₇·10H₂O), and aldehydes, in particular from the compounds of the following formulae: B(OR)₃, RB(OH)₂, B(OH)₃, R-CHO, in which R is chosen from aryls and heteroaryls, and the compounds comprising at least two aldehyde functions, in particular the compounds comprising two aldehyde functions, more particularly the compounds of formula H—C(═O)—(CH₂)ₙ—C(═O)—H, with n ranging from 0 to 6, for example n=0, 1, 2 or 3, the crosslinking agent being for example 3-quinoline boronic acid.

According to another aspect, the present invention also relates to the use of a nano- or microparticle as defined above, or of an assembly of nano- or microparticles as defined above, for the preparation of devices suitable for detection by a giant magnetoresistance sensor.

In all that follows, the embodiments relating to a particle also apply to an assembly of such particles.

According to another aspect, the present invention also relates to a nano- or microparticle comprising a matrix consisting of or comprising at least one polyvinyl alcohol (PVA) and dispersed therein, ferrite, and further comprising an outer layer of silica.

According to a particular embodiment, said outer layer of silica has a thickness from 10 to 150 nm, in particular from 15 to 30 nm, or from 30 to 150 nm.

The presence of this outer layer of silica can allow to keep the cores comprising PVA and ferrite apart, which is likely to give the particles an advantageous stability if required.

According to another aspect, the present invention also relates to a method for preparing a nano- or microparticle as defined above, comprising:
(i) a step of contacting at least one polyvinyl alcohol (PVA) with a ferrite precursor composition A to obtain a composition B;
(ii) a step of heating the composition B to obtain said nano- or microparticles;
(iii)a step of depositing a layer of silica on the nano- or microparticle as obtained at the end of the preceding step by sol-gel synthesis, in particular using a silica precursor chosen from tetraethylorthosilicate and tetramethylorthosilicate, optionally in the presence of aminopropyltriethoxysilane.

According to another aspect, the present invention also relates to a nano- or microparticle comprising a matrix consisting of or comprising at least one polyvinyl alcohol (PVA) and dispersed therein, ferrite, and further comprising an outer layer of silica, which carries on its surface a layer of Al(OH)₃.

According to a particular embodiment, said Al(OH)₃ layer has a thickness from 1 to 10 nm, in particular from 3 to 5 nm.

According to another aspect, the present invention also relates to a nano- or microparticle comprising a matrix consisting of or comprising at least one polyvinyl alcohol (PVA), and dispersed therein, ferrite, and further comprising an outer layer of silica, which is functionalized by compounds carrying carboxylic acid (—COOH) or carboxylic acid anhydride (—C(═O)—O—C(═O)—) groups, in particular compounds:
consisting of or comprising a phosphonic acid in which the phosphorus carries a linear or branched C₁-C₆ alkyl substituted by a carboxylic acid group (—COOH);
consisting of or comprising an arene carrying two carboxylic acid anhydrides (—C(═O)—O—C(═O)—), optionally hydrolysed.

The compounds comprising an arene are likely to be particularly rigid and therefore move the carboxylic acid group away from the surface of the particle, which may allow a particularly efficient coupling if required.

According to a particular embodiment, the present invention relates to a nano- or microparticle comprising a matrix consisting of or comprising at least one polyvinyl alcohol (PVA) and dispersed therein, ferrite, and further comprising an outer layer of silica, which carries on its surface a layer of Al(OH)₃ functionalized in part by compounds carrying carboxylic acid (—COOH) or carboxylic acid anhydride (—C(═O)—O—C(═O)—) groups, in particular compounds:
consisting of or comprising a phosphonic acid in which the phosphorus carries a linear or branched C₁-C₆ alkyl substituted by a carboxylic acid group (—COOH);
consisting of or comprising an arene carrying two carboxylic acid anhydrides (—C(═O)—O—C(═O)—), optionally hydrolysed.

According to a particular embodiment, the present invention relates to a nano- or microparticle comprising an outer layer of silica, which carries on its surface a layer comprising aluminium, in the form of Al(OH)₃ or forming a complex of one of the following formulae:

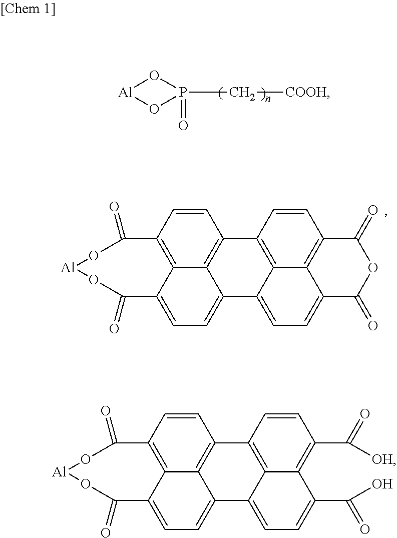

with n ranging from 1 to 6, in particular 1 or 2,
and their mixtures.

According to another aspect, the present invention also relates to a method for preparing a nano- or microparticle as defined above, comprising:
(i) a step of contacting at least one polyvinyl alcohol (PVA) with a ferrite precursor composition A to obtain a composition B;
(ii) a step of heating the composition B to obtain said nano- or microparticles;
(iii)a step of depositing a layer of silica on the nano- or microparticle as obtained at the end of the preceding step by sol-gel synthesis, in particular using a silica precursor chosen from tetraethylorthosilicate and tetramethylorthosilicate, optionally in the presence of aminopropyltriethoxysilane;
(iv) a step of contacting a nano- or microparticle as obtained at the end of the preceding step with an aluminium salt, in particular selected from AlCl₃, Al(NO₃)₃, Al(ClO₄)₃, which are optionally hydrated, the aluminium salt preferably being chosen from AlCl₃, hydrated Al(NO₃)₃ salts and hydrated Al(ClO₄)₃ salts, to obtain a nano- or microparticle comprising an outer layer of silica, which carries on its surface a layer of Al(OH)₃;
(v) a step of contacting the nano- or microparticle obtained at the end of the preceding step with a compound:
   consisting of or comprising a phosphonic acid in which the phosphor carries a linear or branched C₁-C₆ alkyl substituted by a carboxylic acid group (—COOH);
   consisting of or comprising an arene carrying two carboxylic acid anhydrides (—C(═O)—O—C(═O)—);
   for example the following formula:

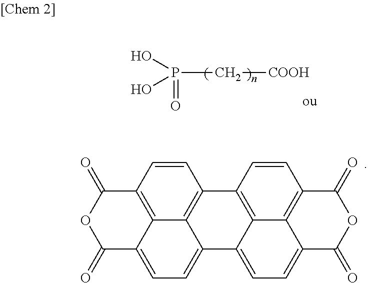

According to another aspect, the present invention also relates to a nano- or microparticle comprising a matrix consisting of or comprising at least one polyvinyl alcohol (PVA) and dispersed therein, ferrite, and further comprising an outer layer of silica, which is functionalized by compounds carrying a macromolecule, in particular a monoclonal antibody,
said nano- or microparticle comprising in particular an outer layer of silica, which is functionalised by compounds carrying a macromolecule, in particular a monoclonal antibody, by means of an amide function formed between a carboxylic acid carried by said compounds and an amine carried by said macromolecule.

According to another aspect, the present invention also relates to a method for preparing a nano- or microparticle as defined above, comprising:
(i) a step of contacting at least one polyvinyl alcohol (PVA) with a ferrite precursor composition A to obtain a composition B;
(ii) a step of heating the composition B to obtain said nano- or microparticles;
(iii)a step of depositing a layer of silica on the nano- or microparticle as obtained at the end of the preceding step by sol-gel synthesis, in particular using a silica precursor chosen from tetraethylorthosilicate and tetramethylorthosilicate, optionally in the presence of aminopropyltriethoxysilane;
(iv) a step of contacting a nano- or microparticle as obtained at the end of the preceding step with an aluminium salt, in particular selected from AlCl₃, Al(NO₃)₃, Al(ClO₄)₃, which are optionally hydrated, the aluminium salt preferably being chosen from AlCl₃, hydrated Al(NO₃)₃ salts and hydrated Al(ClO₄)₃ salts, to obtain a nano- or microparticle comprising an outer layer of silica, which carries on its surface a layer of Al(OH)₃;
(v) a step of contacting the nano- or microparticle obtained at the end of the preceding step with a compound:
consisting of or comprising a phosphonic acid in which the phosphor carries a linear or branched C₁-C₆ alkyl substituted by a carboxylic acid group (—COOH);
consisting of or comprising an arene carrying two carboxylic acid anhydrides (—C(═O)—O—C(═O)—);
for example the following formula:

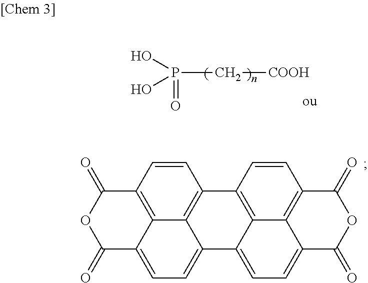

(vi) a step of forming an amide function between the carboxylic acid groups carried by the nano- or microparticle as obtained at the end of the preceding step and an amine carried by said macromolecule, in particular by the formation of intermediate activated esters from said carboxylic acids, for example using 1-ethyl-3-(3-dimethylaminopropyl)carbodiimide (EDC).

According to a particular embodiment, step (vi) is carried out at a pH from 4 to 5, in particular around 4.5.

According to a particular embodiment, the preparation methods as described above are partially or totally carried out in the absence of magnetic fields. More particularly, the steps described above, in particular step (ii) as described above, are carried out in the absence of magnetic fields.

By “absence of magnetic fields”, it is meant in particular the fact of not approaching a magnet, in particular not using magnetic stirring or magnetised devices such as a magnetised autoclave.

According to another aspect, the present invention also relates to a nano- or microparticle as defined above, or an assembly of nano- or microparticles as defined above, for its use in the diagnosis, in particular for the detection of bacteria, in particular in clinical samples without preculture, for example of the whole blood or other biological fluids; or of cancer cells.

The bacteria are in particular pathogenic bacteria well known to the person skilled in the art, in particular of environmental and/or public health interest, for example Yersinia enterocolitica (which can be responsible in particular for gastroenteritis and food poisoning) and Legionella pneumophila (which can be responsible in particular for pneumonia caused by inhalation of microdroplets).

### Definitions

As used in this description, the term “about” refers to a range of values within±10% of a specific value. For example, the term “about 20” comprises the values of 20±10%, i.e., the values of 18 to 22.

For the purposes of this description, the percentages refer to percentages by mass in relation to the total mass of the formulation, unless otherwise stated.

As understood here, the value ranges in the form of “x-y” or “from x to y” or “between x and y” include in particular the x and y bounds as well as the integers between these bounds. For example, “1-5”, or “from 1 to 5” or “between 1 and 5” refer in particular to the integers 1, 2, 3, 4 and 5. The preferred embodiments include each individual integer in the value range, as well as any sub-combination of those integers. For example, the preferred values for “1-5” may comprise the integers 1, 2, 3, 4, 5, 1-2, 1-3, 1-4, 1-5, 2-3, 2-4, 2-5, etc.

As used herein, the term “alkyl” designates a linear- or branched-chain alkyl group having the number of carbon atoms indicated before said term, in particular 2 to 6 carbon atoms, such as ethyl, propyl, isopropyl, butyl, isobutyl, sec-butyl, tert-butyl, pentyl, isoamyl, neopentyl, 1-ethylpropyl, 3-methylpentyl, 2,2-dimethylbutyl, 2,3-dimethylbutyl, hexyl, etc. Thus, an expression such as “C1-C4 alkyl” designates an alkyl radical containing from 1 to 4 carbon atoms. The same applies to the term “alkane”.

Cycloalkyls are in particular alkyls (as defined above) comprising a cycle. One example is cyclohexyl.

As used herein, the term “arene” refers to a substituted or unsubstituted mono- or bicyclic aromatic hydrocarbon cyclic system having 6 to 32 carbon atoms in the cycle. The examples include benzene, naphthalene, anthracene, perylene, chryene, corannulene, phenanthrene, triphenylene, benzo[a]pyrene, coronene, tetracene, pentacene, pyrene and ovalene. The preferred arenes comprise benzene, naphthalene and perylene, unsubstituted or substituted. The definition of “arena” includes condensed cyclic systems, including, for example, the cyclic systems in which an aromatic cycle is condensed to a cycloalkyl cycle. The examples of such condensed cyclic systems comprise, for example, the indane, the indene and the tetrahydronaphthalene.

As used herein, the term “heteroarene” refers in particular to an aromatic group containing 5 to 10 carbon atoms in the cycle, in which one or more carbon atoms of the cycle are replaced by at least one heteroatom such as —O—, —N— or —S—. Examples of heteroarene groups are pyrrole, furan, thiene, pyrazole, imidazole, thiazole, isothiazole, isoxazole, oxazole, oxathiole, oxadiazole, triazole, oxatriazole, furazane, tetrazole, pyridine, pyrazine, pyrimidine, pyridazine, triazine, indole, isoindole, indazole, benzofuran, isobenzofuran, purine, quinazoline, quinole, isoquinole, benzimidazole, benzothiazole, benzothiophene, thianaphthene, benzoxazole, benzisoxazole, cinnoline, phthalazine, naphthyridine and quinoxaline. The definition of “heteroarene” includes the fused cyclic systems, including, for example, the cyclic systems in which an aromatic cycle is fused to a heterocycloalkyl cycle. Examples of such fused cycle systems comprise phthalamide, phthalic anhydride, indoline, isoindoline, tetrahydroisoquinoline, chroman, isochroman, chromene and isochromene.

### BRIEF DESCRIPTION OF THE DRAWINGS

FIG. 1 is a scanning electron microscopy (SEM) image of the dry particles obtained in Example 1, as obtained by depositing an aqueous solution of said particles (mother solution) on a silicon plate, followed by SEM analysis.

FIG. 2 is a scanning electron microscopy (SEM) image of the particles obtained in example 1, at a smaller scale than that shown in FIG. 1.

FIG. 3 shows the extraction of the magnetic moment from commercial particles (micromod 50 nm beads, ademtech 100 nm beads, ademtech 200 nm beads, micromod 500 nm beads and dyna-Myone 1000 nm beads) and particles according to the invention (example 1 and example 3).

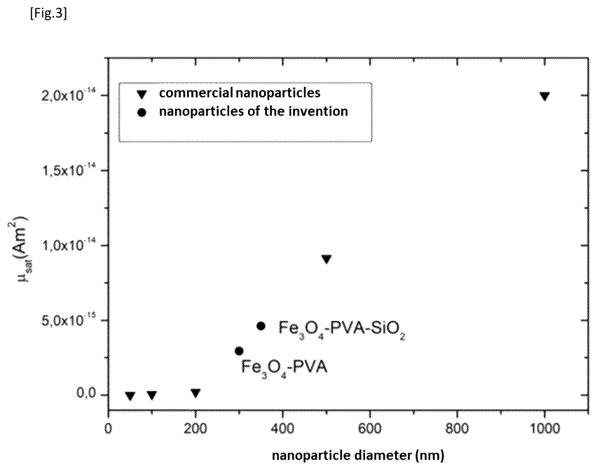

### EXAMPLES

### Example 1: Synthesis of Fe₃O₄-PVA Beads

1 g of PVA is added to 100 ml of DI water. The reaction medium is heated to 80° C. for approximately 1 hour, until the PVA is completely dissolved. The solution is clear and colourless. The temperature was allowed to fall to 50° C. and then 4.065 g of FeCl₃, 6H₂O was added. The dissolution is total. 4.05 g of FeCl₃, 6H₂O is then added. The solution is then translucent red.

At the same time, 8.82 g of sodium citrate 2H₂O and 2.7 g of urea are added to 150 ml of DI water in another container.

The two solutions were mixed at room temperature for 10 minutes, the mixture was orange in colour and the medium cloudy. The resulting suspension is then poured into a Teflon-lined autoclave. 50 ml DI water is added. The autoclave is then heated to 200° C. for 24 hours. After 24 hours, the autoclave is left to cool. The reaction medium is a black suspension. The precipitate is recovered by centrifugation and washed 3 times with DI water, each wash being followed by a centrifugation cycle.

Approximately 2.2 g of dry matter is recovered (after one night at 100° C. in a ventilated oven). The powder is then recovered in 100 ml of anhydrous EtOH and centrifuged again. The precipitate is dried again overnight at 100° C. 1.9 g of powder is recovered. The atomic absorption of the powder gives a mass fraction of 34% Fe. This corresponds to a Fe₃O₄ mass fraction of 47.36%.

The resulting particles have a density of 1.87 kg·m⁻³ compared with 5.17 kg·m⁻³ for Fe₃O₄.

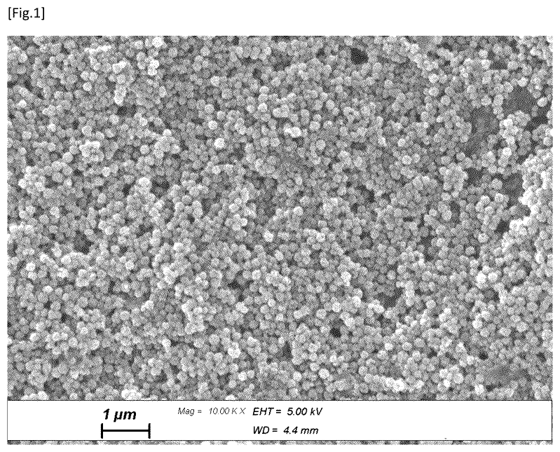

The SEM imaging (FIG. 1) allows to reveal the monodispersity in size of composite particles of the invention obtained in this way. This image also shows the absence of aggregation of the particles of the invention within their mother solution (a medium for biological use), and therefore their stability, which is particularly advantageous, in said medium. Indeed, if there had been aggregation in solution, this would also have been observed after evaporation of the solution when the SEM image was taken.

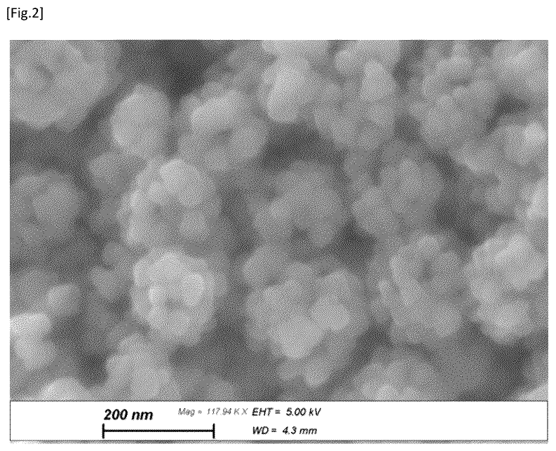

The SEM imaging also allows to highlight the monodispersity in size of the ferrite particles in a composite particle of the invention (FIG. 2).

### Example 2: Magnetic Properties of the Particles of the Invention

The magnetic particles were characterised by measuring magnetisation as a function of the magnetic field at room temperature. It was thus possible to determine the magnetic moment of a magnetic particle of the invention and to compare it with that of commercial magnetic particles of equivalent size (ademtech beads of 200 nm in diameter and micromod beads of 500 nm in diameter). The magnetic moment obtained for the magnetic particles of the invention (according to example 1 or 3) have a moment equivalent to that of the commercial beads.

### Example 3: Functionalisation of Fe₃O₄-PVA Particles According to the Invention

With mechanical stirring, 1 g of particles such those obtained in example 1 are dispersed in 200 ml of EtOH abs. The PVA in the composite is compacted in this anhydrous medium. Then, with stirring, 0.5 g of a mixture of tetraethylorthosilicate and aminopropyltriethoxysilane in a TEOS/APTES molar ratio=20/1 is added (the APTES is likely to act as a catalyst to initiate the polymerisation of the silica layer). After 10 minutes of homogenisation, 1 g of H₂O is stirred. The medium was then left to stir mechanically for 12 hours at room temperature. The product is recovered by centrifugation and washed three times with DI water. The zeta potential of the particles falls to −20-30 mV, illustrating that a layer of silica has been built up on the surface of the particles.

Optionally, the PVA of the Fe₃O₄-PVA core can be cross-linked by reacting a cross-linker of the PVA: for example B(OR)₃, RB(OH)₂, B(OH)₃ or Na₂B₄O₇·10H₂O before growing the silica layer. The advantage of the compounds such as RB(OH)₂ is that, if desired, another organic function can be grafted onto the PVA of Fe₃O₄-PVA: in particular a fluorescent dye. 3-quinoline boronic acid was used successfully: 1 g of Fe₃O₄-PVA particles were dispersed in 200 ml of DI H₂O with mechanical stirring. Then, with stirring, 0.2 g of 3-quinoline boronic acid was added to 50 ml of THF. The medium was then left to stir mechanically for 12 hours at room temperature. The product is recovered by centrifugation, washed three times with DI water, each wash being followed by a centrifugation. The product shows a fluorescence at 540 nm when excited at 400 nm.

The magnetic particles can carry surface carboxylic acid functions, which can then react, for example with EDC to allow the coupling, with a biological molecule.

This can be achieved by changing the zeta potential of the silica layer by hydrolysing an aluminium salt (e.g. AlCl₃, Al(NO₃)₃). Without wishing to limit ourselves to any one theory, the aminopropyltriethoxysilane used as a catalyst is likely to participate in the silica network by leaving pending primary amine functions on which the aluminium salts can hydrolyse to form an Al(OH)₃ layer.

1 g of SiO₂-modified PVA-Fe₃O₄ particles are dispersed in 200 ml of DI H₂O with mechanical stirring. Then 0.3 g of a hydrated aluminium salt is added with stirring. The medium was then left to stir mechanically for 12 hours at room temperature. The product is recovered by centrifugation and washed three times with DI water. The zeta potential of the particles rose to +15 mV, proving that a layer of Al(OH)₃ had been built up on the surface of the particles. Without wishing to limit ourselves to any one theory, the amorphous silica layer on the surface is likely to allow to inhibit the formation of crystallised gibbsite (Al(OH)₃). Without this layer, the gibbsite crystals could start to grow alongside the Fe₃O₄-PVA particles.

The surface layer is then functionalised with functional phosphonic acid grafts, so that the magnetic particles carry pending carboxylic acid functions on their surface.

To do this, 1 g of SiO₂-modified Fe₃O₄-PVA particles with an Al(OH)₃ surface is dispersed in 200 ml of H₂O DI with mechanical stirring. Then 0.2 g of HOOCCH₂PO(OH)₂ and/or HOOC(CH₂)₂PO(OH)₂ is added with stifling. The medium was then left to stir mechanically for 12 hours at room temperature. The product is recovered by centrifugation and washed three times with DI water. The zeta potential of the particles rises to −10 mV, proving that a part of the Al(OH)₃ layer on the surface of the particles has been passivated.

Alternatively, the surface layer can be functionalised with aromatic carboxylic polyanhydrides. For example, the reaction of an aromatic carboxylic acid dianhydride such as perylene-3,4,9,10-tetracarboxylic dianhydride with the surface Al—OH leads to the formation of the following complex:

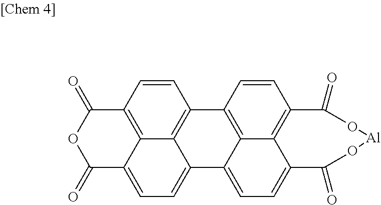

It is a bidentate very firmly attached to the Al(OH)₃ surface.

If the pH of the colloidal suspension becomes acidic, between 4 and 5, the second anhydride function opens to form the following complex, which has two pending carboxylic acid functions from the end of a rigid sp² segment.

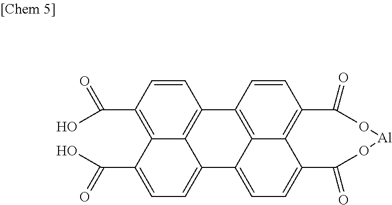

This graft is fluorescent (exc 420 nm, i.e. at low energies, fluoresces in the red). In addition, when the pending carboxyl groups react, the fluorescence varies. It is therefore a probe that allows to evaluate the quality of the EDC coupling as described below.

To do this, 1 g of SiO₂-modified Fe₃O₄-PVA particles with an Al(OH)₃ surface is dispersed in 200 ml of DI H₂O with mechanical stirring. Then, with stifling, 0.1 g of perylene-3,4,9,10-tetracarboxylic dianhydride was added to 15 ml of THF. The medium was then left to stir mechanically for 12 hours at room temperature. The product is recovered by centrifugation and washed three times with DI water. The zeta potential of the particles rose to −5 mV, proving that a part of the Al(OH)₃ layer on the surface of the particles had been passivated. The particles are red.

To functionalize the magnetic beads, the surface carboxyl functions are first modified into activated esters by reaction with EDC. Then, in a second step, in the presence of the primary amines of the lateral chains of the antibodies, an amide bond is formed, ensuring the covalent grafting of the antibodies to the surface of the magnetic beads.

To this end, 80 μL of a solution of EDC (1-ethyl-3-(3-dimethylaminopropyl) carbodiimide) prepared extemporaneously at 4 mg/mL in 0.1 M MES buffer pH 4.7 is added to each milligram of magnetic beads. The mixture is incubated for 10 minutes at 37° C. with stirring at 150 rpm to obtain the intermediate activated esters from the carboxylic acids. Next, 10 to 50 μg of purified antibody are added per mg of beads for conjugation reaction for 2 h at 37° C. with stifling at 150 rpm. Finally, 2 mL of 0.5 mg/mL BSA in 0.1 M MES buffer pH 4.75 is added per mL of functionalised beads. The mixture was incubated for a final 30 minutes at 37° C. with stirring at 150 rpm to ensure the saturation of the non-specific or unreacted sites. The magnetic beads functionalized in this way were washed twice by centrifugation in PBS pH7.4+0.1% BSA and recovered in the latter buffer at a final concentration of 5 mg/mL for a storage at 4° C. until use.

1. A nano- or microparticle comprising a matrix consisting of or comprising at least one polyvinyl alcohol (PVA) and dispersed therein, ferrite.

2. The nano- or microparticle according to claim 1, wherein the polyvinyl alcohol is cross-linked, in particular with the aid of a crosslinking agent chosen from the following compounds: boric acid, boronic acids, boric acid esters, boronic acid esters, borax (Na₂B₄O₇·10H₂O), and aldehydes, in particular from the compounds of the following formulae: B(OR)₃, RB(OH)₂, B(OH)₃, R-CHO, in which R is chosen from aryls and heteroaryls, and the compounds comprising at least two aldehyde functions, in particular the compounds comprising two aldehyde functions, more particularly the compounds of formula H—C(═O)—(CH₂)ₙ—C(═O)—H, with n ranging from 0 to 6, for example n=0, 1, 2 or 3, the crosslinking agent being for example 3-quinoline boronic acid.

3. The nano- or microparticle according to claim 1, wherein:
is shaped like a sphere or spheroid; and/or
has a size from 50 to 1000 nm, in particular from 150 to 450 or 500 nm.

4. The nano- or microparticle according to claim 1, wherein the at least one polyvinyl alcohol is present in an amount of 30 to 50% by mass relative to the total mass of said nano- or microparticle.

5. An assembly of nano- or microparticles according to claim 1, the size distribution of which has a uniformity coefficient from 0.95 to 1 and/or in which more than 95% of these nano- or microparticles have a size of 180 nm +/−10%.

6. A method for preparing a nano- or microparticle according to claim 1, comprising:
(i) a step of contacting at least one polyvinyl alcohol (PVA) with a ferrite precursor composition A to obtain a composition B;
(ii) a step of heating the composition B to obtain said nano- or microparticles.

7. A method of preparation of devices capable of being detected by giant magnetoresistance sensors, said method comprising a step of using nano- or microparticle according to claim 1, or an assembly of nano- or microparticles according to claim 5.

8. The nano- or microparticle according to claim 1, further comprising an outer layer of silica, which is functionalised by compounds carrying a macromolecule, in particular a monoclonal antibody,
said outer layer of silica being functionalised in particular by means of an amide function formed between a carboxylic acid carried by said compounds and an amine carried by said macromolecule.

9. The method of preparing a nano- or microparticle according to claim 8 comprising:
(iii) a step of depositing a layer of silica on the nano- or microparticle according to claim 1 by sol-gel synthesis, in particular using a silica precursor chosen from tetraethylorthosilicate and tetramethylorthosilicate, optionally in the presence of aminopropyltriethoxysilane;
(iv)a step of contacting the nano- or microparticle obtained at the end of the preceding step with an aluminium salt, chosen in particular from AlCl₃, Al(NO₃)₃, Al(ClO₄)₃, which are optionally hydrated, the aluminium salt preferably being chosen from AlCl₃, the hydrated Al(NO₃)₃ salts and the hydrated Al(ClO₄)₃ salts, to obtain a nano- or microparticle comprising an outer layer of silica, which carries on its surface a layer of Al(OH)₃;
(v) a step of contacting the nano- or microparticle obtained at the end of the preceding step with a compound:
consisting of or comprising a phosphonic acid in which the phosphor carries a linear or branched C₁-C₆ alkyl substituted by a carboxylic acid group (—COOH);
consisting of or comprising an arene carrying two carboxylic acid anhydrides (—C(═O)—O—C(═O)—);
for example the following formula: 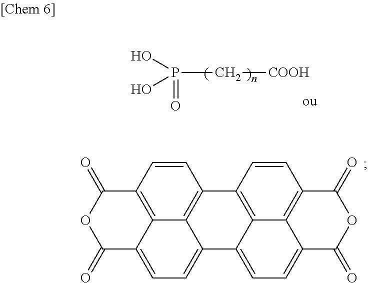
(vi)a step of forming an amide function between the carboxylic acid groups carried by the nano- or microparticle obtained at the end of the preceding step and an amine carried by said macromolecule, in particular by the formation of intermediate activated esters from said carboxylic acids, for example by using 1-ethyl-3-(3-dimethylaminopropyl)carbodiimide (EDC).

10. A method of diagnosis, in particular for the detection of bacteria, in particular in clinical samples without preculture, for example whole blood or other biological fluids; or of cancer cells, said method comprising a step of using nano- or microparticle according to claim.
